(19) United States
(12) Patent Application Publication
(10) Pub. No.: US 2025/0042739 A1
(43) Pub. Date: Feb. 6, 2025

(54) METAL-FREE PROCESS FOR HYDROGEN PEROXIDE PRODUCTION

(71) Applicant: Portland State University, Portland, OR (US)

(72) Inventors: Theresa McCormick, Portland, OR (US); Avik Bhattacharjee, Portland, OR (US)

(73) Assignee: Portland State University, Portland, OR (US)

(21) Appl. No.: 18/924,084

(22) Filed: Oct. 23, 2024

Related U.S. Application Data
(63) Continuation of application No. PCT/US2023/018803, filed on Apr. 17, 2023.
(60) Provisional application No. 63/336,065, filed on Apr. 28, 2022.
(60) Provisional application No. 63/385,245, filed on Nov. 29, 2022.

(51) Int. Cl. C01B 15/026; B01J 31/02; B01J 35/39; C07C 45/29

(52) U.S. Cl. CPC C01B 15/026; B01J 31/0271; B01J 35/39; C07C 45/29; C01P 2002/84; C01P 2002/86

(57) ABSTRACT

Disclosed herein are aspects of a method for producing hydrogen peroxide. The method may comprise exposing a substrate to light in the presence of an organic photocatalyst and oxygen, and optionally a photosensitizer to produce an oxidation product of the substrate and hydrogen peroxide. The reaction may be performed in water and in some aspects, the reaction oxidizes water to produce hydrogen peroxide.

### CROSS REFERENCE TO RELATED APPLICATION

This application is a continuation of PCT Patent Application No. PCT/US2023/018803, filed Apr. 17, 2023, which claims the benefit of the earlier filing dates of U.S. Provisional Patent Application No. 63/336,065, filed Apr. 28, 2022, and U.S. Provisional Patent Application No. 63/385,245, filed Nov. 29, 2022, all of which are incorporated herein by reference in their entireties.

### FIELD

Disclosed herein is a method for producing hydrogen peroxide using a photocatalyst and light, in the absence of a metal.

### BACKGROUND

Oxidation reactions are important for the production of many agro- and pharma-chemicals and often use stoichiometric metal-oxides that produce significant amounts of toxic waste. Using oxygen from the air as the terminal oxidant would eliminate this waste. However, aerobic oxidation reactions are challenging and often require transition-metal catalysts. Nevertheless, aerobic oxidation can be achieved using photocatalysis. Primary and secondary alcohols generated from alkenes, produced from cracking longer chain petrochemical alkanes, can be oxidized to corresponding higher-value aldehydes and ketones.

The current method of H₂O₂ production utilizes the Anthraquinone Process (AP) that uses H₂ and O₂ and a palladium catalyst. This process requires large chemical plants and major disadvantages including mass-transport limitations of hydrogenation and oxidation steps, and substantial investments for purification, storage, and distribution. Only a handful of plants produce over 4.5 million tons of H₂O₂ that are used industrially every year, which is then shipped to the point-of-use.

### SUMMARY

Disclosed herein are aspects of a method, comprising exposing a substrate to light in the presence of an organic photocatalyst and oxygen, such as oxygen in the air, at a temperature of 50° C. or less, and in the absence of a metal. In some aspects, the temperature is from −10° C. to 50° C., such as from 0° C. to 40° C., or from 0° C. to 25° C.

The method may be a method for producing hydrogen peroxide and/or producing an oxidation product of the substrate. The method may comprise a mixture comprising the substrate, the photocatalyst, and a solvent system, to the light in the presence of the oxygen. The solvent system may comprise a polar solvent, such as an alcohol, acetonitrile, water, or a combination thereof. In some aspects, the solvent system comprises acetonitrile and water.

In certain aspects, the substrate and the solvent system are both water.

In other aspects, the substrate is a primary or secondary alcohol, a primary amine, a thiol, an aromatic alkane, an alkene, or a compound comprising a carbonyl moiety with a methylene adjacent to the carbonyl moiety. In some aspects, the substrate is aliphatic, and may be a primary or secondary aliphatic alcohol. In other aspects, the substrate is aromatic, and may be selected from

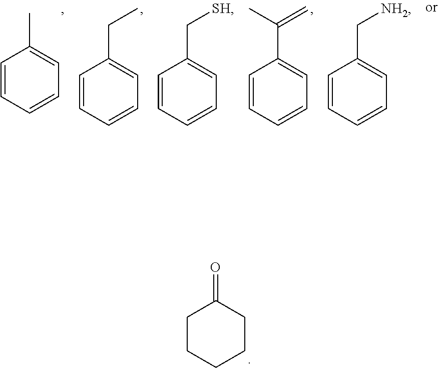

Or alternatively, the substrate may have a formula

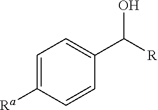

wherein R is H, CH₃ or optionally substituted phenyl and R^{a} is H, C₁₋₆alkyl, halogen, CN, CF₃, —OC₁₋₆alkyl.

In any aspects, the photocatalyst may comprise a

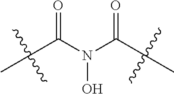 [SMILES: *C(=O)N(O)C(*)=O]

moiety. In some aspects, the photocatalyst has a structure according to Formula I

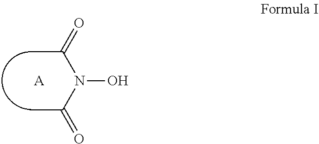

where ring A is a 5- or 6-membered non-aromatic ring optionally fused to an aromatic ring system. Ring A may be a 5-membered ring according to Formula II

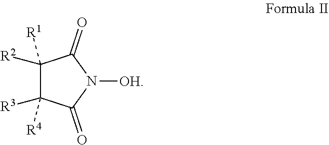

With respect to Formula II, each of R¹, R², R³, and R⁴ is H; and R¹ and R⁴ are H, and R² and R³ together form a double bond; or R¹ and R⁴ are absent, as indicated by the dashed lines, and R² and R³ together with the atoms to which they are attached, form an optionally substituted aromatic ring. And in some aspects, the photocatalyst has a structure according to Formula III

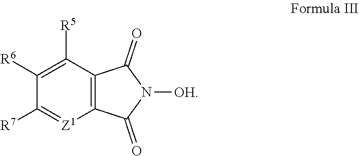

With respect to Formula III, Z¹ is N or C(R⁸); and each of R⁵, R⁶, R⁷ and R⁸ independently is H, —O(C₁₋₆alkyl), or halogen, or two adjacent groups from R⁵, R⁶, R⁷ and R⁸, together with the atoms to which they are attached, form a heterocyclic ring optionally substituted with oxo (═O) and/or —OH.

In other aspects, ring A is a 6-membered ring having a formula IV

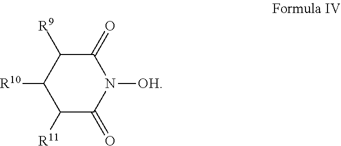

With respect to Formula IV, each of R⁹, R¹⁰, and R¹¹ are H; or R⁹, R¹⁰, and R¹¹ are fused to an aromatic bicyclic ring.

In certain aspects, the photocatalyst is

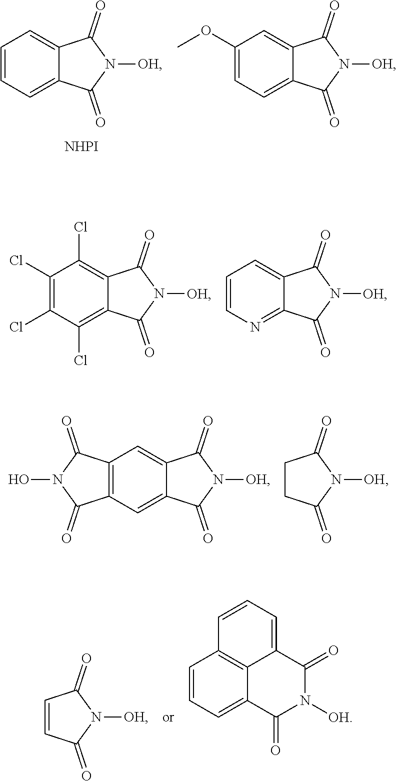

And in particular aspects, the photocatalyst is NHPI.

The method may further comprise exposing the substrate and the photocatalyst to a photosensitizer, such as Rose Bengal, methylene blue, Eosin B, Ru(bpy)₃, methyl green, rubrene, a fullerene, a fluorene, a nanoparticle, or a combination thereof.

In any aspects, the light may be visible light, and may be white light, such as sunlight. Additionally, or alternatively, the light may comprise ultraviolet light.

Also disclosed herein is a method comprising exposing a mixture comprising a compound comprising a benzylic alcohol moiety, a N-hydroxyphthalimide catalyst, and a photosensitizer to visible light to form an oxidized product of the compound and hydrogen peroxide. The method also may form benzoin. The mixture may be exposed to the visible light in the presence of oxygen gas. The visible light may be white light or monochromatic light. In some aspects, the light is LED light. In some aspects, the photosensitizer is a singlet oxygen photosensitizer, and/or the N-hydroxyphthalimide catalyst is N-hydroxyphthalimide.

In any aspects, the compound comprising a benzylic alcohol moiety may have a structure

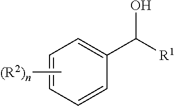

wherein R¹ is H, aliphatic, aryl or heteroaryl; each R² independently is aliphatic, aryl, heteroaryl, halogen, OH, NO₂, CO₂H, or ester; and n is 0, 1, 2, 3, 4, or 5.

In some aspects, the mixture further comprises a solvent, and/or the catalyst is present in the mixture in an amount of from 1 mol % to 10 mol %. And in any aspects, the method may further comprise separating the hydrogen peroxide from the oxidized product of the compound and any unreacted components from the mixture.

A method for producing hydrogen peroxide also is disclosed herein. In some aspects, the method comprises exposing a mixture comprising a compound comprising a benzylic alcohol moiety, NHPI, and a photosensitizer to visible light to form a reaction mixture comprising hydrogen peroxide; and separating the hydrogen peroxide from the reaction mixture. The compound comprising a benzylic alcohol moiety may be

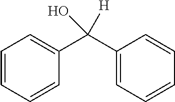 [SMILES: OC(c1ccccc1)c1ccccc1]

and/or the photosensitizer may be Rose Bengal.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a schematic chemical synthesis illustrating the anthraquinone process for hydrogen peroxide production.

FIG. 2 is a schematic diagram illustrating a possible mechanism for the aerobic oxidation of alcohols through photosensitization, where the excited photosensitizer (PS*) creates singlet oxygen (¹O₂) which initiates the formation of PINO which then results in oxidation of the alcohol.

FIG. 3 is a schematic diagram illustrating an alternative mechanism for the aerobic oxidation of alcohols through photocatalyzation, where irradiation of NHPI leads to homolytic cleavage of the NO—H bond and formation of PINO.

FIG. 4 is a schematic diagram illustrating the catalytic reaction cycle of alcohol oxidization and hydrogen peroxide production using NHPI as a catalyst.

FIG. 5 is a ¹H NMR spectrum acquired in CD₃CN using 400 MHz spectrometer for the reaction mixture of the photochemical aerobic oxidation of benzhydrol in presence of an internal standard (ethylene carbonate, ‘s’) after 3 days of irradiation time. The peaks a, b, c, d, and e correspond to the starting material whereas the peaks f, g, and h are for the oxidation products benzophenone and H₂O₂.

FIG. 6 is an enlarged image of the aromatic region of the NMR spectrum in FIG. 5. The peaks marked as c, d, and e are the aromatic protons of benzhydrol starting material, whereas peaks f, g, and h correspond to the aromatic protons of benzophenone product.

FIG. 7 is a ¹H NMR spectrum acquired in CD₃CN using 400 MHz spectrometer for the reaction mixture of the photochemical aerobic oxidation of benzyl alcohol in presence of an internal standard (ethylene carbonate, ‘s’) after 3 days of irradiation time. The peaks a, b and c correspond to the starting material whereas the peaks f, g, h and i are for the oxidation products benzaldehyde, H₂O₂, and benzoin.

FIG. 8 is an enlarged image of the aromatic region of the NMR spectrum in FIG. 7. The peaks marked as f, g, and h correspond to the aromatic protons of benzaldehyde and the other peaks correspond to the condensation product, benzoin.

FIG. 9 is a graph of absorbance versus wavelength, illustrating the UV-Vis spectra of the Ti-porphyrin dye.

FIG. 10 is a graph of yield of benzophenone versus time, illustrating the yield of benzophenone with 1 day of irradiation, followed by 1 day in the dark, and 1 day irradiated. The yields were determined by ¹H NMR, reaction contained diphenylmethanol (5 mmol), NHPI (5 mol %) Rose Bengal, (1×10⁻⁴ M) in 5 mL acetonitrile (ACN) under white light irradiation at room temperature.

FIG. 11 is a stacked ¹H NMR plot of the aromatic region of the benzhydrol oxidation reaction mixture taken in CD₃CN before irradiation with white light (t=0 days) through 18 days of irradiation.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing disclosed aspects from discussed prior art, the aspect numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

“Aliphatic” describes a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀) from one to six (C₁₋₆), or from one to four carbon atoms (C₁₋₄) for an acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety.

“Alkane” refers to an acyclic branched or unbranched hydrocarbon having the general formula CₙH₂ₙ₊₂, and consisting entirely of hydrogen atoms and saturated carbon atoms.

“Alkene” refers to an acyclic branched or unbranched hydrocarbon having one or more carbon-carbon double bond.

“Alkyl” refers to a saturated aliphatic hydrocarbyl group having from 1 to 15 (C₁₋₁₅) or more carbon atoms, more typically 1 to 10 (C₁₋₁₀) carbon atoms such as 1 to 6 (C₁₋₆) carbon atoms or 1 to 4 (C₁₋₄) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂—CH₂CH₂CH₃), isobutyl (—CH₂CH₂(CH₃)₂), sec-butyl (—CH(CH₃)(CH₂CH₃), t-butyl (—C(CH₃) 3), n-pentyl (—CH₂CH₂CH₂CH₂CH₃), and neopentyl (—CH₂C(CH₃) 3).

“Hydroxy” refers to an —OH moiety. A primary hydroxy is a hydroxy on a —CH₂OH group. A secondary hydroxy is a hydroxy on a —CH(OH)— moiety.

“Amine” refers to a —NR² moiety where each R independently is H, alkyl, or aryl. Primary amine refers to a —NH₂ moiety.

“Aromatic” refers to a cyclic, conjugated group or moiety of, unless specified otherwise, from 5 to 15 ring atoms having a single ring (e.g., phenyl, pyridinyl, or pyrazolyl) or multiple condensed rings in which at least one ring is aromatic (e.g., naphthyl, indolyl, or pyrazolopyridinyl), that is at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

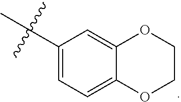 [SMILES: *c1ccc2c(c1)OCCO2]

An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety.

“Aromatic alkane” refers to a compound comprising an aromatic moiety, such as an aryl or heteroaryl moiety, substituted with an alkyl moiety. Example aromatic alkanes include, but are not limited to, methylbenzene (toluene), dimethylbenzene (e.g., ortho-, meta- and para-xylene), ethylbenzene, and methylpyridine (e.g., 2-, 3- and/or 4-substituted).

“Carbonyl” refers to a C═O moiety in an organic compound.

“Halogen” refers to fluoro, chloro, bromo or iodo.

“Methylene” refers to a —CH₂— moiety in an organic compound.

“Photocatalyst” refers to a compound or material that absorbs light and provides the energy to a reacting substance to facilitate a chemical reaction. A photocatalyst may raise the energy of the catalyst to a higher energy level to facilitate the chemical reaction.

“Photosensitizer” refers to a compound that absorbs light and transfers the energy from the incident light to another compound. A photosensitizer may generate a reactive oxygen species (ROS). A photosensitizer may itself be oxidized or reduced and then participate in electron transfer with another compound. In some aspects, a photosensitizer absorbs light energy and transfers the energy to a ground state triplet oxygen (³O₂) to make excited state singlet oxygen (¹O₂). In this case, the reactive oxygen species is the singlet oxygen (¹O₂).

“Thiol” refers to an organic compound comprising an —SH moiety.

### II. Overview

Oxidation reactions to transform alcohols to carbonyls often produce large quantities of hazardous waste by using stoichiometric oxidants such as chromium, and manganese reagents. Also, it is important to develop catalysts that will allow oxidation reactions to use O₂, (preferably from air) as the stoichiometric oxidant. Traditionally, efforts in developing aerobic oxidation catalysts have used homogeneous transition-metal-catalysts. Since a goal of the disclosed technology is to develop an oxidation catalyst that can simultaneously oxidize a substrate and produce H₂O₂, reactions must avoid any metals to prevent decomposition of the H₂O₂ produced by the reaction.

Preliminary results demonstrated that alcohols can be photocatalytically oxidized to carbonyl compounds using an organo-photocatalyst, N-Hydroxyphthalimide (NHPI). This process simultaneously converts O₂ to H₂O₂. Oxidizing industrially relevant alcohols to high-value products using a metal-free photocatalyst is a novel approach to address aerobic oxidation reactions while the photochemical production of H₂O₂ has potential industrial applications. NHPI as an organic oxidation catalyst is usually used under thermal-condition with metal-activators and follows a radical mechanism. First, homolytic cleavage of the NO—H bond generates a phthalimide-N-oxyl (PINO) radical. PINO then reacts with the substrates to generate carbon-centered radicals that eventually react with O₂ to form a meta-stable hydroxy(perhydroxy) intermediate. Finally, hydroperoxyl leaves as H₂O₂ generating a carbonyl compound as the final-product. However, cobalt and/or manganese co-catalysts are typically used to initiate H-atom abstraction for NHPI to produce the active radical species, PINO. Thus, any H₂O₂ produced in these systems decomposes under the reaction-conditions. Furthermore, thermal-reactions require elevated temperatures (typically 70-90° C.) where PINO is prone to decomposition. Accordingly, this method does not allow both the oxidized product and H₂O₂ to be isolated and often no recoverable H₂O₂ is produced. If a transition metal catalyst or co-catalyst is used in the reaction then H₂O₂ is not recoverable as a product because the metal completes the 4H⁺/4e⁻ reduction of O₂ to water.

Triplet-photosensitizers can generate the reactive-oxygen-species ¹O₂ with relatively low-energy light. It has been reported that NHPI can be used under photocatalytic conditions. The photocatalytic oxidation of the α-C of unsaturated hydrocarbons was reported where NHPI was used as a radical organocatalyst. (Zhao et al. Photocatalytic Oxidation of α-C—H Bonds in Unsaturated Hydrocarbons through a Radical Pathway Induced by a Molecular Cocatalyst. ChemSusChem 2019, 12, 2795-2801.) However, in this work, CdS acts as a photo-redox catalyst to generate PINO and to reduce oxygen to superoxide. This method therefore uses a potentially toxic metal catalyst. The main focus of this disclosure is to establish an oxidation method of activated alkanes to corresponding aldehydes or ketones or carboxylic acids, and discloses H₂O formation as a byproduct, and not H₂O₂.

Similarly, it has been demonstrated that the excited-state of graphitic-carbon-nitride (g-C₃N₄) can activate O₂ to superoxide. (Zhang, et al. Visible-Light-Induced Metal-Free Allylic Oxidation Utilizing a Coupled Photocatalytic System of g-C₃N₄ and N-Hydroxy Compounds. Adv. Synth. Catal. 2011, 353, 1447-1451.) Again, the superoxide promotes hydrogen abstraction from NHPI, generating PINO that oxidizes toluene. This differs from the proposed reaction mechanism discussed herein where ¹O₂ is the reactive oxygen species. And the paper does not disclose or suggest hydrogen peroxide production.

And Rose Bengal has been used as a photosensitizer for the synthesis of β-oxy alcohols. (Zhang et al., Visible Light-Induced Aerobic Dioxygenation of α,β-Unsaturated Amides/Alkenes toward Selective Synthesis of β-Oxy Alcohols Using Rose Bengal as a photosensitizer. Org. Chem. Front. 2021, 8, 2215-2223.) However, the reaction disclosed by this paper is not catalytic since PINO is consumed, thereby increasing the cost and lowering the commercial usefulness of the reaction.

The method disclosed herein is distinguished from these publications because the method uses an organic catalyst, optionally with an organic photosensitizer, for a metal-free reaction that facilitates hydrogen peroxide production and isolation.

The papers discussed above disclose NHPI as photocatalyst. However, none of these papers report hydrogen peroxide production and/or isolation, as the main focus of these papers are photocatalytic aerobic oxidations. Some use metal-containing co-catalysts or additive that would decompose any hydrogen peroxide that may be produced in the reaction.

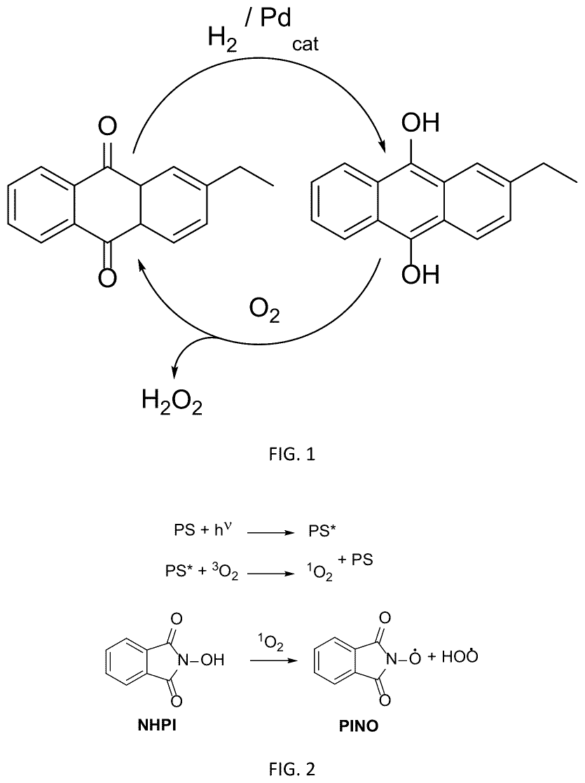

H₂O₂ has widespread use, including for the oxidation of petrochemicals. H₂O₂ is currently produced through the Anthraquinone Process (AP) (FIG. 1). Briefly, 2-ethylanthraquinone is reduced by H₂ gas over a palladium catalyst. The solution is moved to a separate reactor where auto-oxidation with O₂ regenerates 2-ethylanthraquinone and produces H₂O₂, which is removed in an aqueous wash. The benefit of this method is that dangerous H₂/O₂ mixtures are avoided. However, the drawbacks include a purification step that is energy intensive. And additionally, the catalysts need to be frequently replaced, and reactor design limits point-of-use production. Furthermore, the required H₂ gas is produced by steam reforming fossil fuels, which increases carbon emissions.

Direct synthesis of H₂O₂ that use H₂ and O₂ have an ideal atom-economy. However, mixtures of H₂ and O₂ are explosive at most concentrations, and the majority of catalysts that activate H₂ also activate decomposition of H₂O₂. Therefore, most systems rely on rare earth metal catalysts such as iridium, platinum or palladium.

Production of H₂O₂ from water oxidation or O₂ reduction is desirable because it eliminates the need for explosive gas mixtures, and both water and O₂ are readily available. Coupled to a photochemical reaction, this is a “green” method for H₂O₂ production that does not use metal catalysts that are often toxic and/or difficult to extract from the earth, does not require gas that is produced by steam reforming fossil fuels, and can be produced more easily at a point-of-use, thereby reducing the environmental impact of both the reaction itself and subsequent transportation. Disclosed herein is a method for producing H₂O₂ by photo-oxidization of alcohols from water and O₂ without a need for a metal catalyst or thermal activation.

### III. Reaction

Disclosed herein are aspects of a method for producing hydrogen peroxide without the use of a metal catalyst. In some aspects, the method comprises exposing a substrate to light in the presence of an organic photocatalyst and oxygen, and optionally a photosensitizer. The reactants and reagents do not include a metal, and therefore do not include a metal-containing photocatalyst or a metal-containing photosensitizer. Therefore, the reaction proceeds in the absence of a metal.

The light may be natural light, artificial light, or a combination thereof. In some aspects, the light is white light, that is it contains substantially all of the visible wavelengths of light, and may further include infrared (IR) and/or ultraviolet (UV) light. In other aspects, the light contains only certain wavelengths, such as certain color wavelengths of the visible spectrum, and/or may or may not include IR and/or UV. In some aspects, UV-Vis light is used.

The reaction is monitored with peroxide-test-strips to identify and roughly quantify the presence of hydrogen peroxide. And the reaction mixture and reaction products are analyzed by IR and/or ¹³C and ¹H-NMR to identify and quantify oxidation products including hydrogen peroxide. Hydrogen peroxide is further quantified using titrimetric (e.g., iodometry) and spectroscopic (e.g., ammonium vanadate) methods, or by quantitative ¹H NMR.

### A. Substrate

Substrates suitable for use in the disclosed method include any organic compound that comprises an oxidizable chemical functionality with an alpha hydrogen. For example, an organic compound comprising a primary or secondary alcohol (including a primary or secondary aromatic alcohol or a primary or secondary aliphatic alcohol), a primary amine, a thiol, particularly a primary or secondary thiol, an aromatic alkane, an alkene, a compound comprising a carbonyl moiety with a methylene adjacent to the carbonyl moiety, or water. A person of ordinary skill in the art understands that an aromatic substrate is a substrate where the oxidizable chemical functionality is either aromatic or is alpha to an aromatic moiety, for example, benzyl alcohol or the methyl group in toluene. An aliphatic substrate is a substrate where the oxidizable chemical functionality is an aliphatic moiety, and/or is not alpha to an aromatic moiety, for example, ethanol or isopropanol. That is, in an aromatic substrate the carbon with the alpha hydrogen is either in the aromatic moiety or is directly attached to the aromatic moiety, whereas in an aliphatic substrate, the carbon with the alpha hydrogen is not in an aromatic moiety or directly attached to an aromatic moiety. Example substrate compounds include, but are not limited to,

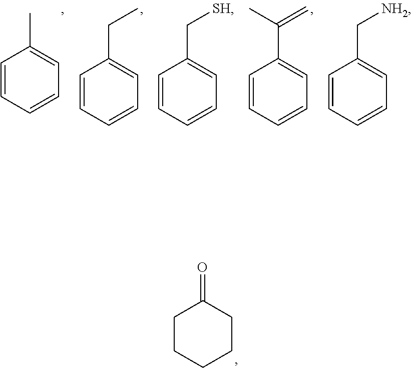

or a compound having a formula

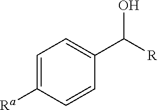

where R is H, CH₃ or optionally substituted phenyl, and R^{a} is H; C₁₋₆alkyl, such as CH₃; halogen, such as Cl; CN; CF₃; or —OC₁₋₆alkyl, such as OMe.

### B. Solvent

The reaction proceeds in a solvent system suitable to dissolve both the substrate and the catalyst. In some aspects, the solvent system comprises a polar solvent. Exemplary suitable polar solvents include, but are not limited to, water, acetonitrile, alcohol (for example, methanol or ethanol), fluorinated solvent (for example, 2,2,2-trifluoroethanol (TFE), 1,1,1,3,3,3-hexafluoroisopropanol (HFIP), perfluoro-tert-butanol, 1,3-bis(1,1,1,3,3,3-hexafluoro-2-hydroxypropyl)benzene, or 2-fluoroethanol (MFE)), or a combination thereof. In some aspects, a non-polar solvent might be used, either alone or in combination with one or more polar solvents. Exemplary non-polar solvents include, but are not limited to, ethyl acetate, and chlorinated solvents, such as dichloromethane, or dichloroethane.

In one aspect, acetonitrile is used as the solvent system.

In another aspect, water is used as the solvent system.

And in a further aspect, a combination of water and acetonitrile is used as the solvent system, such as from 4:1 acetonitrile:water, to 1:4 acetonitrile:water.

### C. Temperature

The reaction proceeds at a temperature suitable to facilitate the oxidation reaction. A suitable temperature is any temperature between the freezing point and boiling point of the solvent system. In some aspects, the temperature is selected that does not substantially decompose the catalyst.

In certain aspects, the reaction proceeds at a temperature of 50° C. or below, such as from −10° C. to 50° C., from 0° C. to 40° C., from 0° C. to 30° C., or from 0° C. to 25° C.

### D. Photocatalyst

The organic photocatalyst is a catalyst that does not contain a metal. Typically, the catalyst comprises a

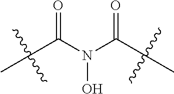 [SMILES: *C(=O)N(O)C(*)=O]

moiety. In some aspects, the catalyst has a structure according to general Formula I

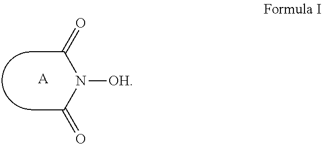

With respect to Formula I, ring A is a 5- or 6-membered non-aromatic ring, optionally fused to an aromatic ring system, such as a single or bicyclic aromatic ring system.

In some aspects, ring A is a 5-membered ring according to Formula II

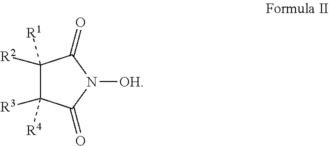

With respect to Formula II, each of R¹, R², R³, and R⁴ is H; R¹ and R⁴ are H, and R² and R³ together form a double bond; or RI and R⁴ are absent, as indicated by the dashed lines, and R² and R³ together with the atoms to which they are attached, form an optionally substituted aromatic ring. In certain aspects, the compound has a structure according to Formula III

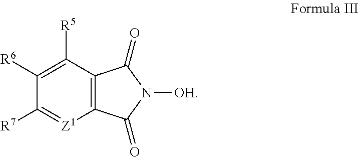

With respect to Formula III, Z¹ is N or C(R⁸), and each of R⁵, R⁶, R⁷ and R⁸ independently is H, —O(C₁₋₆alkyl), or halogen, such as Cl, Br, F or I, typically, Cl, or two adjacent groups, such as R⁵ and R⁶, R⁶ and R⁷, or R⁷ and R⁸, together with the atoms to which they are attached, form a heterocyclic ring optionally substituted with oxo (═O) and/or —OH groups. In some aspects the heterocyclic ring is

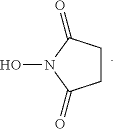

In some aspects, R⁶ and R⁷ form the heterocyclic ring, and each of R⁵ and R⁸, if present, independently is H, —O(C₁₋₆alkyl), or halogen, typically, H.

In other aspects, each of R⁵, R⁶, R⁷ and R⁸, if present, independently is H, —O(C₁₋₆alkyl), or halogen, such as Cl, Br, F or I, typically, Cl. In some aspects, each of R⁵, R⁶, R⁷ and R⁸, if present, is H or Cl. In other aspects, one of R⁵, R⁶, R⁷ and R⁸, if present, is-O(C₁₋₆alkyl), such as methoxy, and the rest are H. In one aspect, R⁶ is —O(C₁₋₆alkyl), such as methoxy, and R⁵, R⁷ and R⁸, if present, are H.

In one aspect, Z¹ is N, and R⁵, R⁶, and R⁷ are as defined above. In some aspects, R⁵, R⁶, and R⁷ are H.

In another aspect, Z¹ is C(R⁸), and R⁵, R⁶, R⁷ and R⁸ are as defined above.

In alternative aspects, ring A is a 6-membered ring having a formula IV

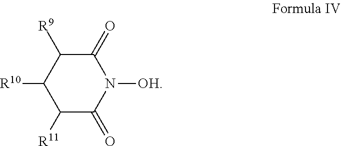

With respect to Formula IV, each of R⁹, R¹⁰, and R¹¹ are H or are fused to an aromatic bicyclic ring, such as naphthalene.

Exemplary compounds according to the disclosed formulas include, but are not limited to:

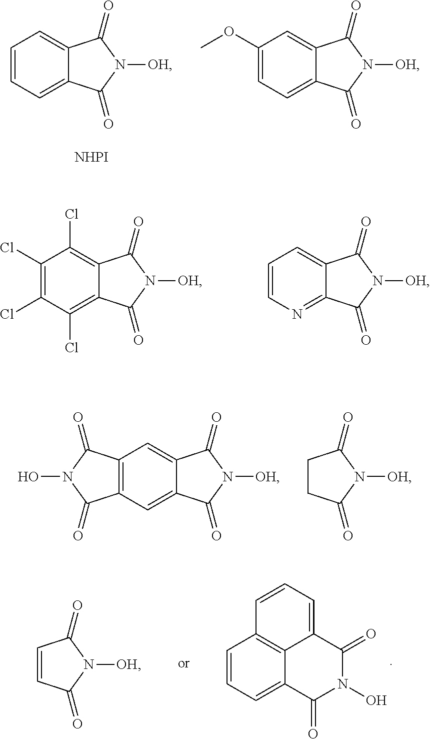

### E. Photosensitizer

Photosensitizers suitable for use in the disclosed method include any photosensitizer that can transfer energy to ground state triplet oxygen upon suitable irradiation, such as visible light irradiation and/or near IR light (NIR). In some aspects, a photosensitizer is selected to be soluble in the solvent system, and/or to be unreactive with respect to singlet oxygen and the reagents. In other aspects, the photosensitizer is selected to facilitate singlet oxygen formation upon exposure to particular wavelengths of light, such as particular colors in the visible spectrum or NIR light. Suitable photosensitizers include, but are not limited to, Rose Bengal, methylene blue, Eosin B, Ru(bpy)₃, methyl green, rubrene, a fullerene (alkyl, aryl, alkoxy-substituted or unsubstituted) a fluorene (e.g. comprising 9,9-substituted, or 2,7-substituted fluorene) a nanoparticle (e.g. CdTe, ZnSe, SiNP, CNP, AuNP, BiNP, or nanoparticles comprising encapsulated small molecule triplet photosensitizers that are attached or encapsulated by silica, proteins, or polymers), or a combination thereof.

### IV. Applications

The disclosed method provides an environmentally friendly process for hydrogen peroxide production. Using light as the energy source and not using potentially toxic metal catalysts significantly reduces the environmental impact of the oxidation process, especially when the process is carried out on an industrial scale. This process could be used at industrial scale for hydrogen peroxide production, or on a small scale to produce hydrogen peroxide for a specific use, including in situ hydrogen peroxide. Additionally, oxidizing water to produce hydrogen peroxide using light as the energy source provides an environmentally friendly process for hydrogen peroxide production that does not require the use of potentially toxic metal catalysts.

The disclosed method also provides an environmentally friendly process for oxidation of substrates using oxygen as the oxidant. This method does not use stoichiometric metal oxides and does not produce toxic waste byproducts.

### V. Examples

### Example 1

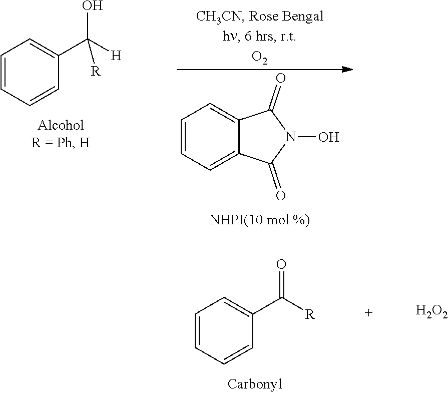

Acetonitrile containing Rose Bengal, 5 M alcohol, and 10 mol % NHPI was irradiated at room-temperature with white LEDs for 6 hours under oxygen-atmosphere. Water and toluene were added, and presence of H₂O₂ was detected in the aqueous-layer using peroxide-test-strips. Absorption spectroscopy with a selective Ti-dye (Oxo[5,10,15,20-tetra(4-pyridyl)porphyrinato]titanium(IV)) confirmed the presence of H₂O₂ by a decrease in the absorption at 432 nm. The identity of the oxidized product was confirmed with IR-spectroscopy. This result demonstrated that this system oxidizes benzylic alcohols to aldehydes and ketones with the concomitant production of H₂O₂.

Several control-experiments were conducted (Table 1). All reactions were run for 6 hours in 5 mL of acetonitrile with benzyl alcohol, if included. The reactions were performed in presence and absence of photosensitizer, and in both cases H₂O₂ was produced (Entry 1 and 2). However, if the reaction was run with no catalyst (NHPI) (Entry 3-5), no alcohol (Entry 6), or no oxygen (Entry 7) then no H₂O₂ was detected (Table 1).

TABLE 1

| | [Alcohol] | [NHPI] | | | Temp | | |
|---|---|---|---|---|---|---|---|
| Entry | (mmol) | (mmol) | O₂ | Light | (° C.) | PS | H₂O₂ Test |
| 1 | 5 | 0.5 | + | white | 25 | + | + |
| 2 | 5 | 0.5 | + | white | 25 | − | + |
| 3 | 5 | − | + | white | 25 | + | − |
| 4 | 5 | − | + | white | 25 | − | − |
| 5 | 5 | − | + | − | 25 | − | − |
| 6 | − | 0.5 | + | white | 25 | + | − |
| 7 | 5 | 0.5 | − | white | 25 | + | − |
| 8 | 5 | 0.5 | + | − | 25 | − | − |
| 9 | 5 | 0.5 | + | − | 25 | + | − |
| 10 | 5 | 0.5 | + | white | 0 | + | + |
| 11 | 5 | 0.5 | + | − | 0 | + | − |
| 12 | 5 | 0.5 | + | green | 25 | + | + |
| 13 | 5 | 0.5 | + | green | 25 | − | − |

At room-temperature, and in the dark, the reaction also did not proceed (Entry 8 and 9). Previously it was suggested that NHPI could be used as a thermal-catalyst at 75° C. for oxidation. To rule-out the possibility of the reaction being heated by irradiation experiments were conducted in an ice-bath. H₂O₂ was still detected at this temperature (Entry 10-11). Finally, the reaction-mixture was irradiated with green-light instead of white-light to selectively excite the photosensitizer. The reaction proceeded only if there is photosensitizer present (Entry 12 and 13).

### Discussion

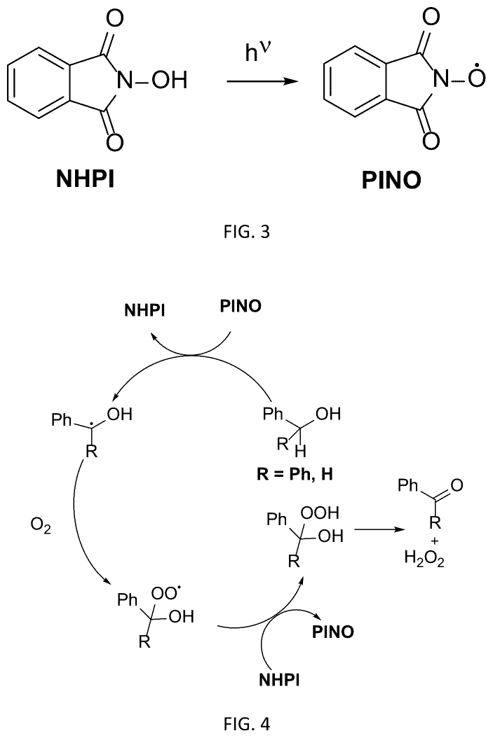

These preliminary results suggested that NHPI was acting as an organo-photocatalyst to simultaneously oxidize benzylic alcohols and produce H₂O₂. Control experiments confirmed that the system was light-driven, and oxygen was the only oxidant. However, H₂O₂ was formed with and without the photosensitizer using white-light, and only with the photosensitizer present when irradiated with green-light. This suggested that two mechanisms were occurring (FIGS. 2 and 3). Without being bound to a particular theory, one possible mechanism was photosensitized and proceeded through the singlet-oxygen production. The other possible mechanism was photocatalytic, and involved direct excitation of NHPI. This hypothesis was supported by the UV-Vis absorption spectra for NHPI that showed an absorption peak at 293 nm with a tail out to 360 nm that could be directly excited with the white-light (data not shown). Since the dissociation energy of the O—H bond is 88.1 kcal/mol, excitation into the low-energy absorption band of NHPI would be energetic enough to break this bond.

These preliminary results supported the hypothesis that metal-free photocatalytic aerobic oxidation of alcohols can be used to produce H₂O₂ along with high value oxidation products.

### Example 2

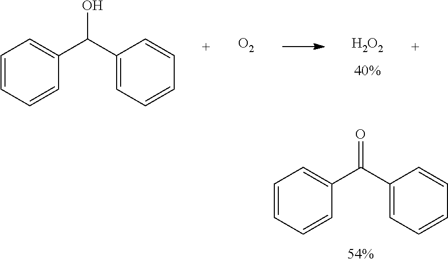 [SMILES: OC(c1ccccc1)c1ccccc1 ; "O2" ; "H2O2" ; O=C(c1ccccc1)c1ccccc1]

5 mmol Benzylic alcohol (benzhydrol) was dissolved in 5 mL acetonitrile containing 1×10⁻⁴ M Rose Bengal in presence of 5 mol % catalyst (NHPI). The solution was stirred (1500 rpm standard magnetic stirrer) under oxygen atmosphere (reaction flask was sealed with a balloon filled with oxygen gas) and white LED (12 V, 6000 K) at room temperature. The % yield after three days of irradiation was 54% (ketone) and 40% (H₂O₂), as determined by ¹H-NMR.

In a second run of the reaction, the % yield was 73% (ketone) and 57% (H₂O₂).

### Example 3

Ethanol or water also can be used as the electron donor in the disclosed method. In a trial, acetonitrile containing Rose Bengal, 5 M ethanol or water, and 10 mol % NHPI was irradiated at room-temperature with white LEDs for 12 hours under oxygen-atmosphere. After a workup, the presence of H₂O₂ was detected in the aqueous-layer using peroxide-test-strips for both reactions run in water and ethanol. Absorption spectroscopy with a selective Ti-dye (Oxo[5,10,15,20-tetra(4-pyridyl)porphyrinato]titanium(IV)) confirmed the presence of H₂O₂ by a decrease in the absorption at 432 nm. ¹H NMR spectroscopy also is used to identify the presence of H₂O₂ and quantify the amount of H₂O₂ in the reaction mixture.

### Example 4

H₂O₂ has widespread use in several laboratory and industrial processes and has many household uses. Recent reports reveal that H₂O₂ can also act as a sustainable, carbon-neutral fuel in an electrochemical fuel cell, producing only water as the waste material. However, the current method of H₂O₂ production utilizes the energy intensive Anthraquinone Process that uses H₂ and O₂ and an expensive Pd-catalyst. Disclosed herein is a simple strategy to achieve aerobic oxidation of benzylic alcohols (for example, benzhydrol, and benzyl alcohol) in metal-free and photocatalytic conditions and the concomitant reduction of oxygen to H₂O₂. Coupled to a photochemical reaction, this is a green method for H₂O₂ production.

Oxidation reactions using NHPI follows a radical mechanism. Homolytic cleavage of the NO—H bond generates the phthalimide-N-oxyl (PINO) radical (Scheme 1). PINO reacts with substrate to generate a benzylic radical that reacts with O₂ to form a metastable hydroxy (perhydroxy) intermediate. Finally, hydroperoxyl leaves as H₂O₂ generating a carbonyl compound as the final product. However, under thermochemical conditions cobalt and/or manganese co-catalysts are often used to initiate H-atom abstraction from NHPI to produce the active radical species, PINO. Thus, any H₂O₂ produced in these systems decompose under these reaction-conditions, and to the inventors' knowledge, no attempts to isolate H₂O₂ from the reaction have been reported. Furthermore, thermal reactions require elevated temperatures (70-90° C.) where PINO is prone to decomposition. Hence, utilizing low-temperature photochemical reactions without a metal co-catalyst should allow both the oxidized products and H₂O₂ to be isolated.

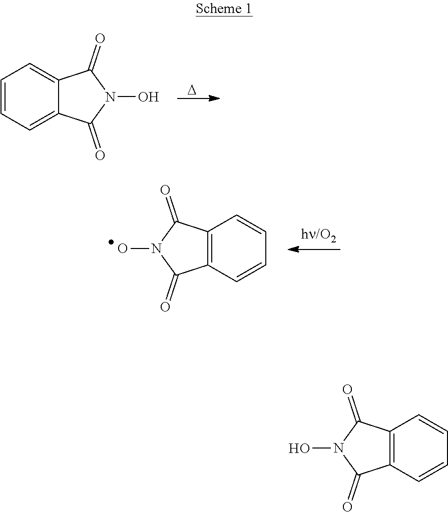

Photocatalytic oxidation of the α-C of unsaturated hydrocarbons has been reported with NHPI as a radical organo-catalyst where CdS acts as a photo-redox catalyst to generate PINO and to reduce oxygen to superoxide. Similarly, the excited-state of graphitic-carbon-nitride (g-C₃N₄) can activate O₂ to superoxide, which promotes hydrogen abstraction from NHPI, generating PINO. Photocatalysis through PINO generation has also been reported for heterogenous systems using α-Fe₂O₃ or TiO₂. This differs from the reaction mechanism disclosed herein where ¹O₂ is the ROS. In a recent paper by Zhang et al., (Org. Chem. Front., 2021, 8, 2215-2223) Rose Bengal was used as a photosensitizer for the synthesis of β-oxy alcohols. However, this reaction is not catalytic since PINO is consumed during the reaction.

### Experimental

### Experimental Methodologies

All chemicals used in this study were purchased from Tokyo Chemical Industries (TCI).

### Photochemical Oxidation of Benzyl Alcohols

In a 10 mL round bottom flask equipped with a stir bar, benzhydrol (5 mmol) was dissolved in a 5 mL solution of Rose Bengal (1×10⁻⁴ M) in acetonitrile. Solid NHPI (0.25 mmol) was added to the solution. The flask was sealed with a balloon filled with oxygen gas. The contents of the flask were stirred (1500 rpm) while being irradiated with a ring of white LEDs (Solid Apollo 24 W 16 ft) at room temperature for 72 hours.

Extraction of Hydrogen Peroxide (H₂O₂)

The reaction mixture (5 mL) was mixed with toluene (5 mL) in a separatory funnel. The organic layer was extracted with water (5 mL). The presence of H₂O₂ was confirmed in the aqueous layer using quantitative peroxide test-strips (MilliporeSigma™ MQuant™).

### Quenching Experiments

In a 10 mL round bottom flask equipped with a stir bar, benzhydrol (5 mmol) was dissolved in a 5 mL solution of Rose Bengal (1×10⁻⁴ M) in acetonitrile. Different quenching agents (5 mmol) and solid NHPI (0.25 mmol) were added to the solution for different experiments. The flask was sealed with a balloon filled with oxygen gas. The contents of the flask were stirred (1500 rpm) while being irradiated with a ring of white LEDs at room temperature for 72 hours.

### Quantification of Products Using ¹H NMR Spectroscopy

500 μL of the reaction mixture was pipetted in a 5 mL round bottom flask and mixed with 500 μL of 1M ethylene carbonate (internal standard) in acetonitrile solution. The acetonitrile was evaporated on a rotary evaporator. The contents of the flask were redissolved in 500 μL of CD₃CN. ¹H NMR was performed using a 400 MHz Bruker (b400) spectrometer.

### Determination of the Presence of Hydrogen Peroxide Using a Ti-Porphyrin Dye

A 5 M solution of [TiO(TPyPH)₄]⁴⁺ was prepared by dissolving 34.03 mg of the dye in 1 L of 0.05 M hydrochloric acid. To 250 μL DI water, 250 μL 4.8 M perchloric acid and 250 μL [TiO(TPyPH)₄]⁴⁺ dye solution was added. The solution was mixed and allowed to stand for 5 minutes at room temperature. This solution was further diluted with 2.5 mL of DI water. The absorbance of this solution was measured. The solution was mixed with a sample of H₂O₂ solution of known concentration, the aqueous extract of the reaction mixture, and a blank. The absorption data for all the solutions were measured using a Shimadzu UV-3600 spectrometer.

Quantification of H₂O₂ by Iodometric Titration.

A 0.5 mL aliquot of a benzhydrol oxidation reaction at 48 hours was extracted using 5 mL of H₂O. 1.2 mmol of potassium iodide and 1 mL of 1 M HCl was added to the aqueous solution. A second solution containing 0.1 M of Na₂S₂O₃ in water was titrated into the first solution that was a brown/black color. When the reaction began to lighten 5 drops of a 1 N starch solution was added. The titration continued until reaction became clear yellow. Analysis done in triplicate yielded 9% hydrogen peroxide after 48 hours.

Generation of Singlet Oxygen without Light.

Benzhydrol, 0.93 g (5.0 mmol) was added to 5 ml of acetonitrile and heated to 40° C. N-hydroxyphthalimide, 0.042 g (0.5 mmol), and Li₂MoO₄, 0.87 g (5 mmol) was added to the solution. The reaction flask was covered using aluminum foil. 2.5 ml (22 mmol) hydrogen peroxide 30% by weight was added in 0.5 ml increments over the course of 5 hours and was stirred for 24 hours. By ¹H NMR, 50% conversion to benzophenone was observed after 24 hours of reaction time. The same reaction conditions with no NHPI showed no oxidized products by NMR after 24 hours.

### Computational Methodologies

All computational modeling was done using Gaussian 09 quantum chemistry package and were run on a HPC cluster. The optimization calculations were done using M06-2X functional and 6-311+G(d) basis sets in PCM acetonitrile solvation model. Normal mode frequency calculations were done on the optimized structures to confirm the convergence to a true stationary point with no imaginary frequencies. Gibbs energy change of the reactions were calculated using Hess' law of constant heat summation.

### Results and Discussion

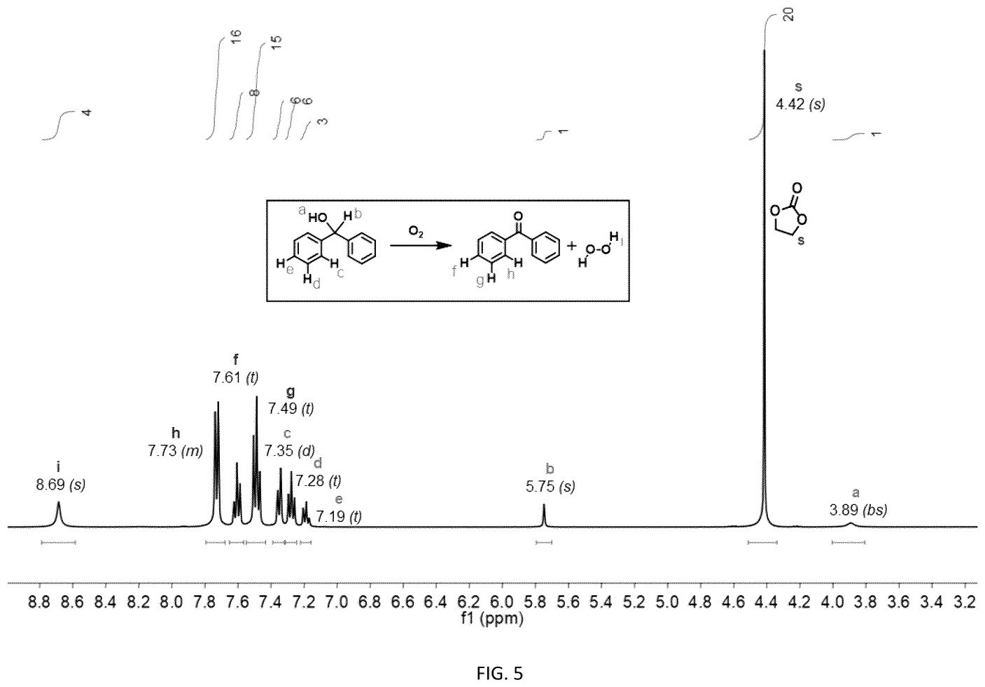

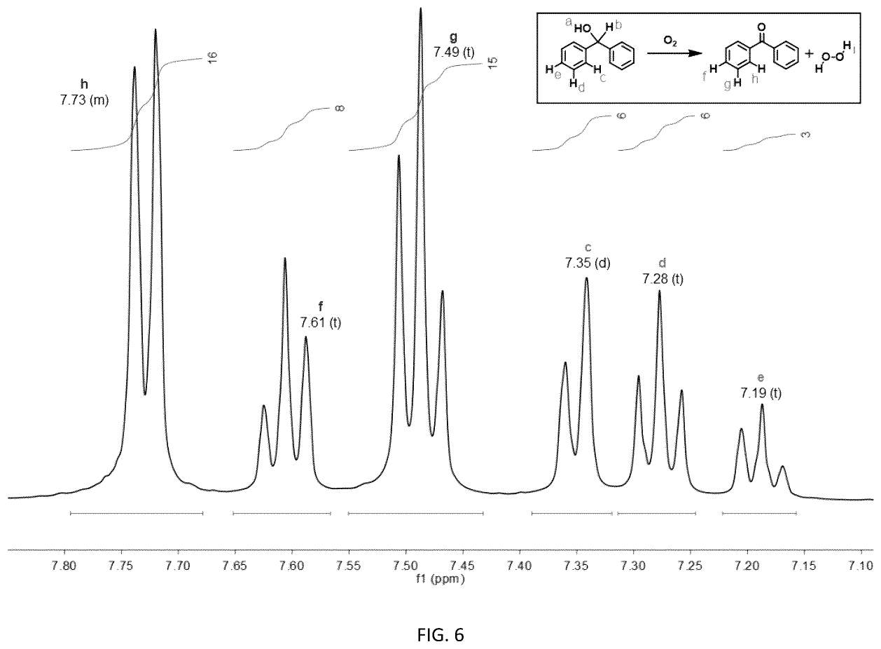

The novel strategy disclosed herein using visible light provided the oxidation products in comparable yield to thermal conditions. Diphenylmethanol (compound 1, 5 mmol) was reacted with molecular O₂ in the presence of NHPI (5 mol %) catalyst and a photosensitizer (Rose Bengal, (RB)) in 5 mL acetonitrile (ACN) under white light irradiation at room temperature for hours to afford benzophenone (compound 3) and H₂O₂ (compound 2) as identified by ¹H NMR (Scheme 2 and FIGS. 5 and 6).

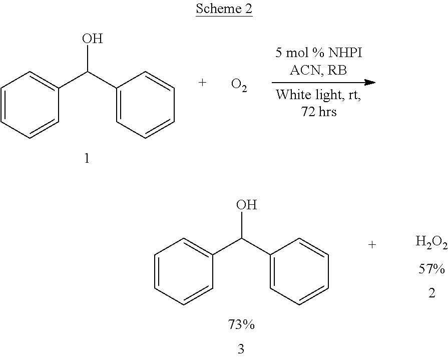

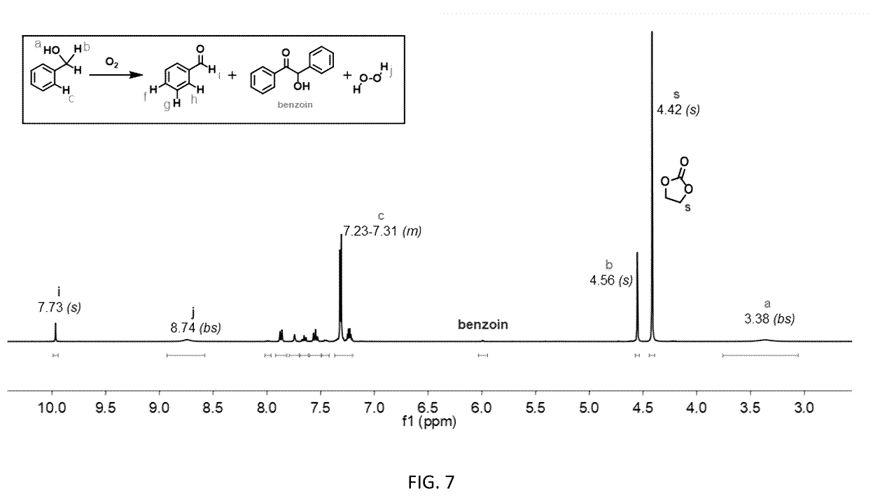

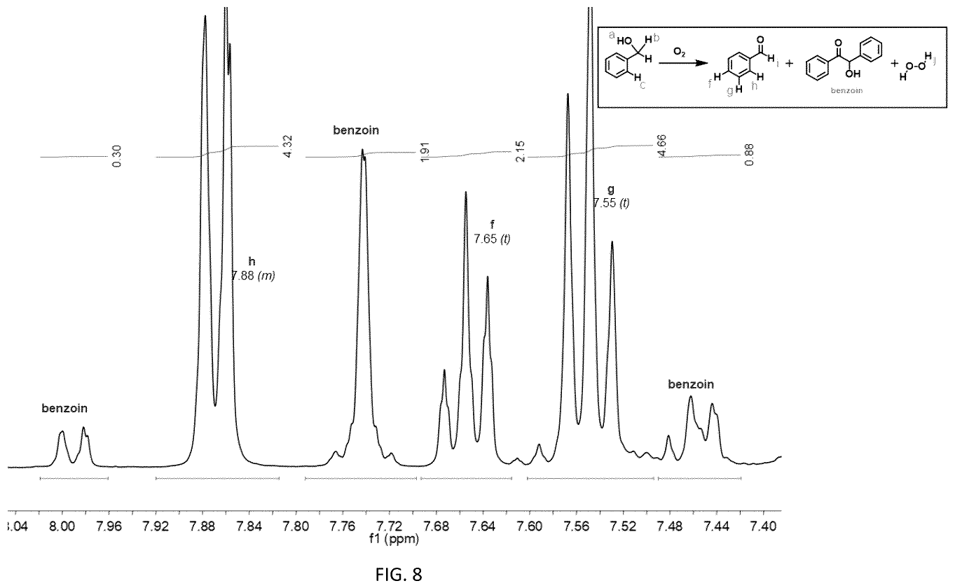

The NMR yields were 73% for benzophenone (compound 3) and 57% for hydrogen peroxide (compound 2). When a primary alcohol, phenylmethanol (compound 4), was oxidized employing the same reaction conditions benzaldehyde (compound 5), 29%; and a trace amount of benzoin, 3% along with H₂O₂ (20%) were identified Scheme 3 and FIGS. 7 and 8).

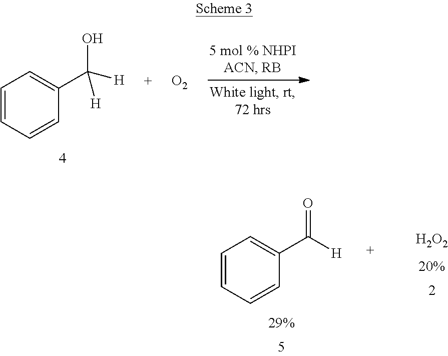

Oxidation of (E)-cinnamyl alcohol and (±)-phenylethyl alcohol resulted in low but measurable yields (5% and 8% respectively, after 72 hours) (Table 2). Reactions were run with 5 mmol alcohol, NHPI (5 mol %) catalyst, and Rose Bengal, (RB) (1×10⁻⁴) in 5 mL acetonitrile (ACN) under white light irradiation at room temperature for 72 hours. The higher yield of the oxidation products for benzophenone can be attributed to the higher stability of the corresponding benzylic radical intermediate.

TABLE 2
Yield of conversion for various alcohols.

| Starting material | 72 hour Yield |
|---|---|
| (E)-Cinnamyl alcohol | 5% |
| (±)-Phenylethyl alcohol | 8% |
| Diphenylmethanol | 39% |
| Phenylmethanol | 29% |

To evaluate the effect of each of the components of the reaction a series of control experiments were run and screened for the production of H₂O₂ using peroxide test-strips (Table 3). Reactions were run for 24 hours with Rose Bengal as the photosensitizer.

TABLE 3
Control experiments by varying the reaction conditions

| | | [NHPI] | | | Temperature | |
|---|---|---|---|---|---|---|
| Entry | [1] mmol^{a} | mol %^{a} | O2ª | Lightª | (° C.) | H₂O₂ test^{b} |
| 1 | 5 | 10 | + | + | 25 | + |
| 2 | 5 | − | + | + | 25 | − |
| 3 | 5 | − | + | − | 25 | − |
| 4 | − | 10 | + | + | 25 | − |
| 5 | 5 | 10 | − | + | 25 | − |
| 6 | 5 | 10 | + | − | 25 | − |
| 7 | 5 | 10 | + | − | 25 | − |
| 8 | 5 | 10 | + | + | 0 | + |
| 9 | 5 | 10 | + | − | 0 | − |

^{a}The ‘+’ sign denotes the presence and the ‘−’ sign denotes the absence of the indicated condition.
^{b}A ‘+’ sign indicates that a blue coloration was seen on the test strip, indicating H₂O₂ production, whereas a ‘−’ sign denotes no color change on the test strip indicating no H₂O₂ production.

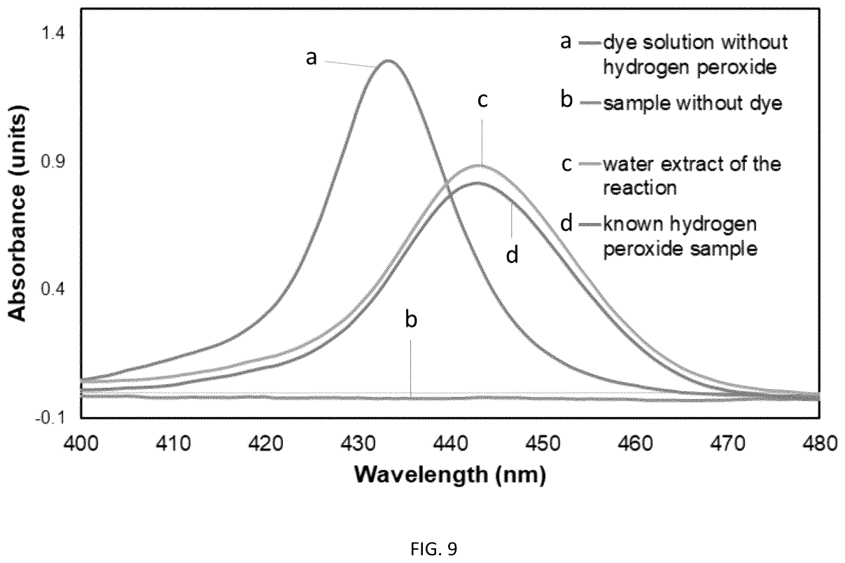

The positive control reaction (Table 3, Entry 1) was run using 5 mmol compound 1, and 10 mol % NHPI in 5 mL of a 104 M RB solution in acetonitrile. The reaction flask was sealed with a balloon filled with oxygen gas. The reaction was stirred vigorously (1500 rpm) for 72 hours while being irradiated with a 24 W white light LED strip coiled in a 12-inch metal tube. Hydrogen peroxide was separated from the organic layer into the aqueous layer by liquid-liquid extraction with 5 mL toluene and 5 mL of water. A blue coloration of the peroxide test-strip indicated the presence of H₂O₂ in the aqueous work-up of the reaction. To rule out a false positive indication on the test strips caused by other types of peroxides or reactive oxygen species (ROS), absorption studies were performed with a selective titanium (IV)-porphyrin dye, oxo[5,10,15,20-tetra(4-pyridyl)porphyrinato]titanium (IV), [TiO(TPyPH₄)]⁴⁺ and a shift was observed in the absorption peak at 432 nm to 445 nm confirming the presence of [TiO₂(TPyPH₄)]⁴⁺ (FIG. 9). With respect to FIG. 9, the UV-Vis spectrum of the Ti-porphyrin dye (a) shows a characteristic absorption band at 432 nm. The characteristic peak was missing in absence of the dye (b). The water extract of the reaction mixture (c), and a hydrogen peroxide solution of known concentration (d) shows a shift in the absorption peak to 445 nm.

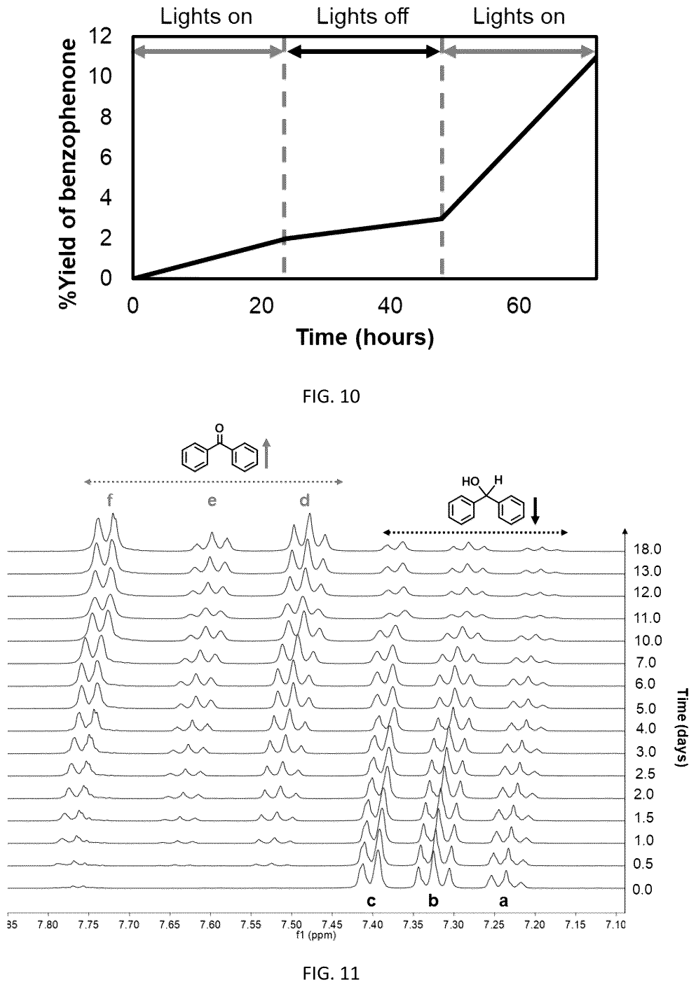

Thus, a color change on the test strip was used to indicate a positive control for the reactions, and if no hydrogen peroxide was detected it was assumed there was no reaction. In absence of NHPI (Entries 2, and 3), or light (Entries 3, 6, and 7) no H₂O₂ was detected. No H₂O₂ was detected if no alcohol was included (Entry 4). To investigate the role of temperature two reactions were ran at 0° C. in the presence, and absence of light. The reaction tested positive for H₂O₂ production at 0° C. in the presence of light (Entry 8), however no H₂O₂ was observed under dark reaction conditions (Entry 9), likewise the reaction didn't proceed without Rose Bengal. The reaction was monitored with irradiation for 1 day, followed by dark for 1 day, and then irradiation for 1 day. The reaction only proceeded while it was irradiated and the overall yield after this 72 hour experiment was similar to only being run for 48 hours, the total time of irradiation (FIG. 10).

The oxidation products (compounds 2 and 3) were found to be stable under the reaction conditions over 18 days by ¹H NMR. The mechanism is shown in Scheme 4 and FIG. 11 provides that ¹H NMR data. With respect to FIG. 11, peaks a, b, and c correspond to the starting material compound 1 and d, e, and f correspond to the product compound 3 in Scheme 4. The intensities of the peaks a, b, and c decreases over time whereas the intensity of the product peaks: d, e, and f are increasing with the progression of the reaction. Around 12 days of irradiation time the reaction reaches the maximum yield at ˜80% of compound 3 and ˜57% of compound 2. Iodometric titration of the extracted aqueous layer gave similar H₂O₂ yields as to those calculated by NMR. Separation and purification of compound 2 resulted in a 74% isolated yield after 7 days of irradiation.

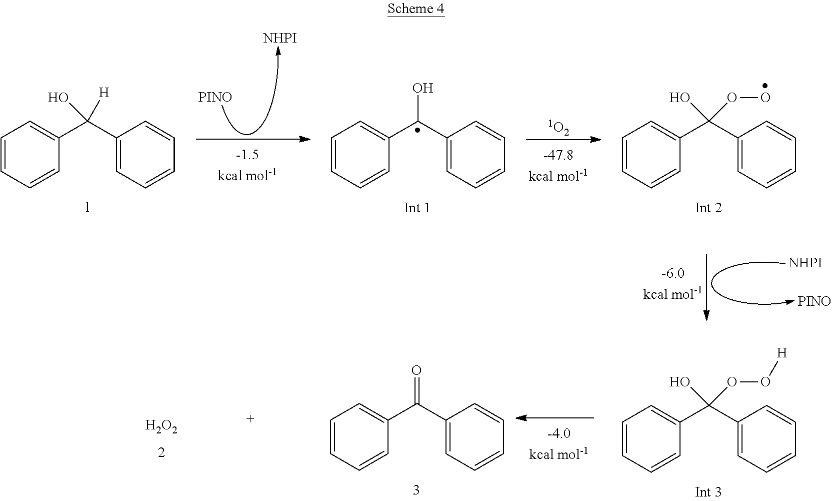

The reaction conditions were optimized by running a three variable full factorial design of experiments (Table 4). The variables considered in this study were concentration of the catalyst (NHPI in mol %), concentration of the photosensitizer (Rose Bengal in molar (RB in M)), and the irradiation time (in days). Using a full factorial design of experiment allows for reaction optimizations with few experiments, and accounts for interacting variables. The % yield was calculated integrating product peaks in the ¹H NMR with respect to an internal standard, ethylene carbonate. After calculating the main and interaction effects of the variables it was observed the yield was maximized at lower levels of catalyst loading (5 mol %) and photosensitizer concentration (1×10⁻⁴ M) but longer irradiation time (3 days).

TABLE 4
Three variable full factorial optimization design for the
photocatalytic aerobic oxidation of diphenylmethanol

| | [NHPI] | | | % Yield | |
|---|---|---|---|---|---|
| Reaction | mol % | [RB] × 10⁻⁴M | Time (days) | 3 | 2 |
| 1 | 10 | 4 | 2 | 10 | 6 |
| 2 | 5 | 1 | 1 | 4 | — |
| 3 | 15 | 1 | 1 | 3 | 1 |
| 4 | 5 | 7 | 1 | 5 | 1 |
| 5 | 15 | 7 | 1 | 5 | 1 |
| 6 | 5 | 1 | 3 | 77 | 50 |
| 7 | 15 | 1 | 3 | 21 | 14 |
| 8 | 5 | 7 | 3 | 26 | 6 |
| 9 | 15 | 7 | 3 | 22 | 15 |

In the disclosed photochemical conditions a lower catalyst-loading produced better performance. This may be due to more stable radical intermediates at lower temperatures minimizing catalyst degradation. The observation that longer irradiation time increases the product yield agrees with the ¹H NMR stability experiments and suggests slower aerobic oxidation kinetics and high energy of activation.

To gain insight on the mechanism of the photocatalytic aerobic oxidation of benzhydrol to benzophenone using NHPI in presence of a photosensitizer, quenching experiments were run with the addition of different scavengers (Q) for intermediates (Table 5).

TABLE 5
Quenching experiments to investigate the nature of the reactive
oxygen species (ROS) and the reaction intermediates

| Entry | Q | Q Type | % + Yield of 3 |
|---|---|---|---|
| 1 | — | — | 77 |
| 2 | NaN₃ | ¹O₂ | 0 |
| 3 | Benzoquinone | O₂⁻ | 12 |
| 4 | (NH₄)₂C₂O₄ | Hole | 4 |
| 5 | ¹BuOH | radicals | 19 |

Addition of sodium azide as a singlet oxygen (¹O₂) scavenger entirely quenched the reaction. No oxidation products were observed after three days of irradiation. Addition of benzoquinone as a superoxide (O₂⁻) scavenger lowered the yield. These two experiments together suggested the primary ROS responsible for the reaction is ¹O₂ but that superoxide may be present and contributing to the reaction yield. This suggested that the Rose Bengal sensitized singlet oxygen as the main ROS. This mechanism was further supported by generating ¹O₂ from Li₂MoO₄ catalyzed decomposition of H₂O₂ without light. This reaction resulted in the oxidation products only in the presence of NHPI.

The suppression of the yield of the oxidation products upon the addition of ammonium oxalate and tert-butyl alcohol suggested that the reaction was dependent on hole and radical intermediates, respectively. This observation further validated the lower yield of oxidation products for the primary alcohol. The benzylic radical intermediate has a lower stabilization for the primary alcohol when compared to the corresponding radical intermediate generated from the secondary alcohol, lowering the overall yield of the reaction.

A previously proposed mechanism using Rose Bengal (RB) and NHPI included reductive quenching of the excited RB*. Subsequent regeneration of RB occurs from RB⁺ getting an electron from deprotonated NHPI created by an added base. Since the disclosed reaction does not have a base to make deprotonated NHPI or an electron donor to regenerate the oxidized RB, this mechanism is not likely under the disclosed conditions. Additionally, using methylene blue (MB) as the photosensitizer resulted in 4.5% production of compound 2 in 72 hours. The lower yield was expected since the singlet oxygen quantum yield of MB is only 0.49 compared to 0.76 for RB. Using this information, it was proposed that the photosensitizer makes singlet oxygen that reacts with NHPI to form PINO that initiates the radical oxidation reaction. The reaction pathway suggested by these experiments was explored using Density Functional Theory (DFT) calculations.

The geometries of the intermediates were optimized using M06-2X/6-311+G(d,p) method implementing an implicit polarized continuum solvation model for acetonitrile in Gaussian 09. Three probable reaction combinations were modeled using ¹O₂ and 302, and 202 to calculate the free energy change associated with the generation of PINO radical from NHPI (ΔG_{r×n}), which is the most crucial step in NHPI catalyzed aerobic oxidation (Table 6). PINO acts as the active catalyst that initiates the radical chain reaction by abstracting the methylene H atom of the secondary alcohol (Scheme 4 compound 1).

TABLE 6
Different reaction combinations and calculated free
energy changes (ΔGrxn) for the formation of PINO

| Entry | Reaction Combinations | ΔGᵣₓₙ (kcal mol⁻¹) |
|---|---|---|
| 1 | ¹NHPI + ³O₂ → ²PINO + ²HO\dot{O} | 27.99 |
| 2 | ¹NHPI + ¹O₂ → ²PINO + ²HO\dot{O} | −9.57 |
| 3 | ¹NHPI + ²O₂⁻ → ²PINO + ²HO\dot{O} | 12.07 |

Unsurprisingly, reactions involving the more energetic ¹O₂ are thermodynamically most favorable, characterized by the most negative free energy change value (Entry 2). However, the less positive Gibbs energy change corresponds to the reaction with superoxide (Entry 3). These results suggested that ¹O₂ can react with NHPI to generate PINO. Direct reaction with ground state ³O₂ was energetically most unfavorable as seen in DFT calculations (Entry 1).

The catalytic mechanism for the aerobic oxidation of compound 1 to compound 3 were modeled using the following steps (Scheme 4). The H atom abstraction by PINO from compound 1 to form the radical intermediate (Int 1) has a free energy of −1.5 kcal mol⁻¹. The second step of the catalytic cycle, formation of the hydroxy (perhydroxy) intermediate (Int 2), was modeled using both ¹O₂ and ³O₂ as oxidants. DFT calculations reveal that formation of Int 2 is thermodynamically more favorable in the presence of ¹O₂ (ΔG_{r×n}=−47.8 kcal mol⁻¹) than ³O₂ (−1.56 kcal mol⁻¹). However, since ¹O₂ is a transient excited state, ³O₂ may be the more likely reactive partner in this step. The computational results suggest either is possible. The metastable hydroxy (hydroperoxy) intermediate (Int 3) formation followed by the release of H₂O₂ (compound 2) to form organic oxidation product 3, have thermodynamic free energies of −6.0 kcal mol⁻¹, and −4.0 kcal mol⁻¹, respectively.

In summary, the use of NHPI to perform aerobic oxidation reactions addresses different challenges in the field of aerobic oxidation catalysis. The disclosed method is a greener alternative to most common alcohol oxidation reactions. Photochemical oxidation can be performed at room temperature or lower. Most importantly, this method generates H₂O₂ as a value-added product. Due to the absence of any metal reactants and mild reaction conditions, the generated H₂O₂ was not decomposed and was extracted from the mixture with simple liquid-liquid extraction procedures. The catalytic mechanism of aerobic oxidation catalyzed by NHPI was explored through quenching experiments and supported by DFT calculations. These results showed the potential of using an organocatalyst that is capable of oxidizing alcohols to H₂O₂ under photochemical conditions, which is of immense interest at both laboratory and industrial scale.

### VI. Exemplary Aspects

The following numbered paragraphs illustrate exemplary aspects of the disclosed technology.

Paragraph 1. A method, comprising exposing a substrate to light in the presence of an organic photocatalyst and oxygen, at a temperature of 50° C. or less, and in the absence of a metal.

Paragraph 2. The method of paragraph 1, wherein the method comprises exposing a mixture comprising the substrate, the photocatalyst, and a solvent system, to the light in the presence of the oxygen.

Paragraph 3. The method of paragraph 1 or paragraph 2, wherein the temperature is from −10° C. to 50° C.

Paragraph 4. The method of paragraph 3, wherein the temperature is from 0° C. to 40° C.

Paragraph 5. The method of paragraph 3, wherein the temperature is from 0° C. to 25° C.

Paragraph 6. The method of any one of paragraphs 2-5, wherein the solvent system comprises a polar solvent.

Paragraph 7. The method of paragraph 6, wherein the polar solvent is an alcohol, acetonitrile, water, or a combination thereof.

Paragraph 8. The method of paragraph 7, wherein the solvent system comprises acetonitrile and water.

Paragraph 9. The method of any one of paragraphs 2-7, wherein the substrate and the solvent system are both water.

Paragraph 10. The method of any one of paragraphs 1-8, wherein the substrate is a primary or secondary hydroxy, a primary amine, a thiol, an aromatic alkane, an alkene, or a compound comprising a carbonyl moiety with a methylene adjacent to the carbonyl moiety.

Paragraph 11. The method of any one of paragraphs 1-9, wherein the substrate is an aliphatic substrate.

Paragraph 12. The method of any one of paragraphs 1-9, wherein the substrate is an aromatic substrate.

Paragraph 13. The method of any one of paragraphs 1-9, wherein the substrate is

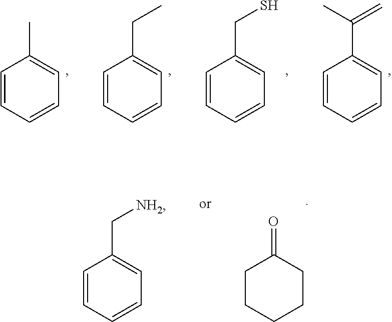

Paragraph 14. The method of any one of paragraphs 1-9, wherein the substrate is a compound having a formula wherein:

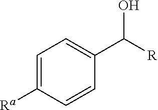

R is H, CH₃ or optionally substituted phenyl; and
R^{a} is H, C₁₋₆alkyl, halogen, CN, CF₃, —OC₁₋₆alkyl.

Paragraph 15. The method of paragraph 14, wherein the substrate is

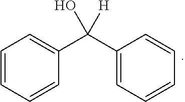 [SMILES: OC(c1ccccc1)c1ccccc1]

Paragraph 16. The method of any one of paragraphs 1-15, wherein the photocatalyst comprises a

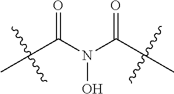 [SMILES: *C(=O)N(O)C(*)=O]

moiety.

Paragraph 17. The method of any one of paragraphs 1-16, wherein the photocatalyst has a structure according to Formula I

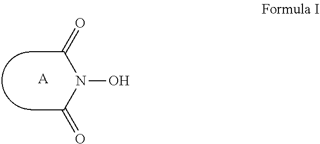

wherein ring A is a 5- or 6-membered non-aromatic ring optionally fused to an aromatic ring system.

Paragraph 18. The method of paragraph 17, wherein ring A is a 5-membered ring according to Formula II

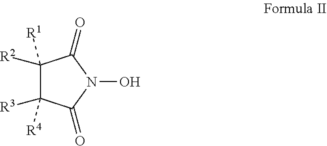

wherein:
each of R¹, R², R³, and R⁴ is H; and
R¹ and R⁴ are H, and R² and R³ together form a double bond; or
R¹ and R⁴ are absent, as indicated by the dashed lines, and R² and R³ together with the atoms to which they are attached, form an optionally substituted aromatic ring.

Paragraph 19. The method of paragraph 17 or paragraph 18, wherein the photocatalyst has a structure according to Formula III

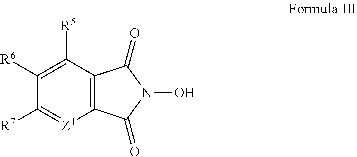

wherein:
Z¹ is N or C(R⁸); and
each of R⁵, R⁶, R⁷ and R⁸ independently is H, —O(C₁₋₆alkyl), or halogen; or two adjacent groups from R⁵, R⁶, R⁷ and R⁸, together with the atoms to which they are attached, form a heterocyclic ring optionally substituted with oxo (═O) and/or —OH.

Paragraph 20. The method of paragraph 17, wherein ring A is a 6-membered ring having a formula IV

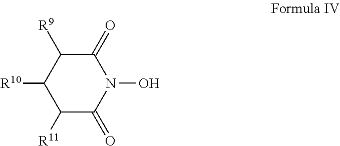

wherein:
each of R⁹, R¹⁰, and R¹¹ are H; or
R⁹, R¹⁰, and R¹¹ are fused to an aromatic bicyclic ring.

Paragraph 21. The method of any one of paragraphs 1-16, wherein the photocatalyst is

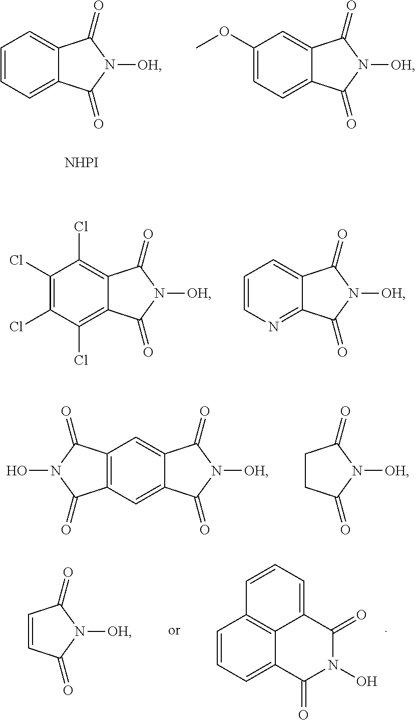

Paragraph 22. The method of paragraph 21, wherein the photocatalyst is

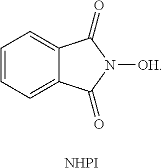 [SMILES: O=C1c2ccccc2C(=O)N1O ; "NHPI"]

Paragraph 23. The method of any one of paragraphs 1-22, further comprising exposing the substrate and the photocatalyst to a photosensitizer.

Paragraph 24. The method of paragraph 23, wherein the photosensitizer is Rose Bengal, methylene blue, Eosin B, Ru(bpy)₃, methyl green, rubrene, a fullerene, a fluorene, a nanoparticle, or a combination thereof.

Paragraph 25. The method of any one of paragraphs 1-24, wherein the light is a visible light.

Paragraph 26. The method of any one of paragraphs 1-25, wherein the light is white light.

Paragraph 27. The method of any one of paragraphs 1-25, wherein the light comprises UV and/or visible light.

Paragraph 28. The method of any one of paragraphs 1-27, wherein the method is a method for producing hydrogen peroxide.

Paragraph 29. The method of any one of paragraphs 1-28, wherein the method is a method for producing an oxidization product of the substrate.

Paragraph 30. A method, comprising exposing a mixture comprising a compound comprising a benzylic alcohol moiety, a N-hydroxyphthalimide catalyst, and a photosensitizer to visible light to form an oxidized product of the compound and hydrogen peroxide.

Paragraph 31. The method of paragraph 30, wherein the mixture is exposed to the visible light in the presence of oxygen gas.

Paragraph 32. The method of paragraph 30 or paragraph 31, wherein the photosensitizer is a singlet oxygen photosensitizer.

Paragraph 33. The method of any one of paragraphs 30-32, wherein the N-hydroxyphthalimide catalyst is N-hydroxyphthalimide.

Paragraph 34. The method of any one of paragraphs 30-33, wherein the compound comprising a benzylic alcohol moiety has a structure

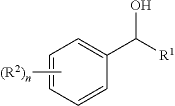

wherein
R¹ is H, aliphatic, aryl or heteroaryl;
each R₂ independently is aliphatic, aryl, heteroaryl, halogen, OH, NO₂, CO₂H, or ester;

and
n is 0, 1, 2, 3, 4, or 5.

Paragraph 35. The method of any one of paragraphs 30-34, wherein the visible light is white light.

Paragraph 36. The method of any one of paragraphs 30-35, wherein the visible light is LED light.

Paragraph 37. The method of any one of paragraphs 30-36, wherein the method also forms benzoin.

Paragraph 38. The method of any one of paragraphs 30-37, wherein the mixture further comprises a solvent.

Paragraph 39. The method of any one of paragraphs 30-38, wherein the catalyst is present in the mixture in an amount of from 1 mol % to 10 mol %.

Paragraph 40. The method of any one of paragraphs 30-39, wherein the method further comprises separating the hydrogen peroxide from the oxidized product of the compound and any unreacted components from the mixture.

Paragraph 41. A method for producing hydrogen peroxide, the method comprising:
exposing a mixture comprising a compound comprising a benzylic alcohol moiety,

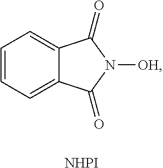 [SMILES: O=C1c2ccccc2C(=O)N1O ; "NHPI"]

and a photosensitizer to visible light to form a reaction mixture comprising hydrogen peroxide; and
separating the hydrogen peroxide from the reaction mixture.

Paragraph 42. The method of paragraph 41, wherein the compound comprising a benzylic alcohol moiety is

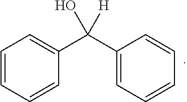 [SMILES: OC(c1ccccc1)c1ccccc1]

Paragraph 43. The method of paragraph 41 or paragraph 42, wherein the photosensitizer is Rose Bengal.

In view of the many possible aspects to which the principles of the disclosed technology may be applied, it should be recognized that the illustrated aspects are only preferred examples of the technology and should not be taken as limiting the scope of the disclosure. Rather, the scope of the technology is defined by the following claims. We therefore claim as our technology all that comes within the scope and spirit of these claims.

We claim:

1. A method, comprising exposing a substrate to light in the presence of an organic photocatalyst and oxygen, at a temperature of 50° C. or less, and in the absence of a metal.

2. The method of claim 1, wherein the method comprises exposing a mixture comprising the substrate, the photocatalyst, and a solvent system, to the light in the presence of the oxygen.

3. The method of claim 1, wherein the temperature is from −10° C. to 50° C.

4. The method of claim 2, wherein the solvent system comprises a polar solvent.

5. The method of claim 1, wherein the substrate is a primary or secondary hydroxy, a primary amine, a thiol, an aromatic alkane, an alkene, or a compound comprising a carbonyl moiety with a methylene adjacent to the carbonyl moiety.

6. The method of claim 1, wherein:
the substrate is 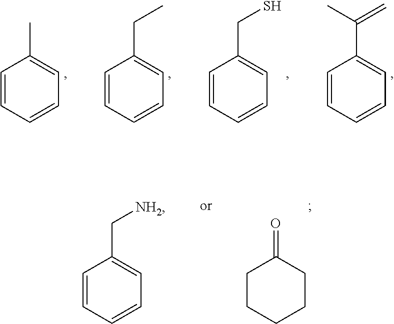
or
the substrate is a compound having a formula 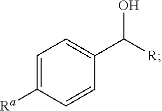
wherein
R is H, CH₃ or optionally substituted phenyl; and
R^{a} is H, C₁₋₆alkyl, halogen, CN, CF₃, —OC₁₋₆alkyl.

7. The method of claim 1, wherein the substrate is

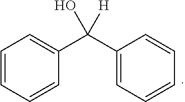 [SMILES: OC(c1ccccc1)c1ccccc1]

8. The method of claim 1, wherein the photocatalyst comprises a 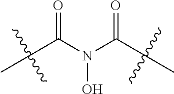 [SMILES: *C(=O)N(O)C(*)=O]
moiety.

9. The method of claim 8, wherein the photocatalyst has a structure according to a formula selected from 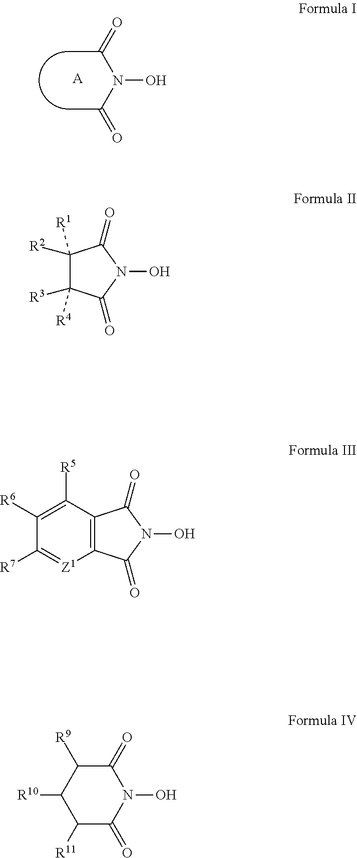
wherein:
ring A is a 5- or 6-membered non-aromatic ring optionally fused to an aromatic ring system;
each of R¹, R², R³, and R⁴ is H; or
R¹ and R⁴ are H, and R² and R³ together form a double bond; or
R¹ and R⁴ are absent, as indicated by the dashed lines, and R² and R³ together with the atoms to which they are attached, form an optionally substituted aromatic ring;
Z¹ is N or C(R⁸);
each of R⁵, R⁶, R⁷ and R⁸ independently is H, —O(C₁₋₆alkyl), or halogen; or
two adjacent groups from R⁵, R⁶, R⁷ and R⁸, together with the atoms to which they are attached, form a heterocyclic ring optionally substituted with oxo (═O) and/or —OH; and
each of R⁹, R¹⁰, and R¹¹ are H; or
R⁹, R¹⁰, and R¹¹ are fused to an aromatic bicyclic ring.

10. The method of claim 8, wherein the photocatalyst is

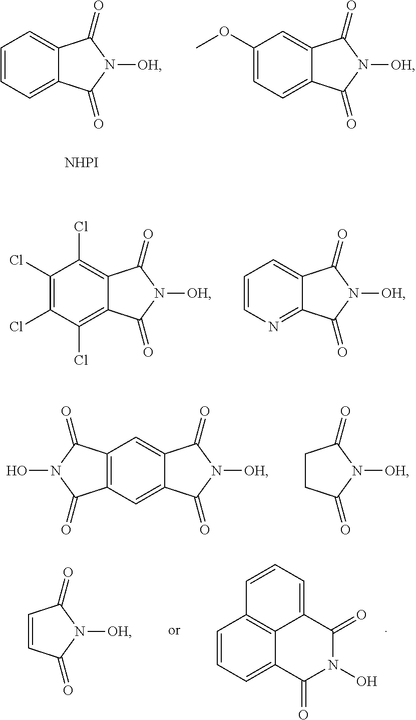

11. The method of claim 8, wherein the photocatalyst is

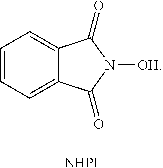 [SMILES: O=C1c2ccccc2C(=O)N1O ; "NHPI"]

12. The method of claim 1, further comprising exposing the substrate and the photocatalyst to a photosensitizer.

13. The method of claim 12, wherein the photosensitizer is Rose Bengal, methylene blue, Eosin B, Ru(bpy)₃, methyl green, rubrene, a fullerene, a fluorene, a nanoparticle, or a combination thereof.

14. The method of claim 1, wherein the light is a visible light.

15. A method, comprising exposing a mixture comprising a compound comprising a benzylic alcohol moiety, a N-hydroxyphthalimide catalyst, and a photosensitizer to visible light, at a temperature of 50° C. or less and in the presence of oxygen gas, and in the absence of a metal, to form an oxidized product of the compound and hydrogen peroxide.

16. The method of claim 15, wherein:
the photosensitizer is a singlet oxygen photosensitizer;
the N-hydroxyphthalimide catalyst is N-hydroxyphthalimide; or
the photosensitizer is a singlet oxygen photosensitizer and the N-hydroxyphthalimide catalyst is N-hydroxyphthalimide.

17. The method of claim 15, wherein the compound comprising a benzylic alcohol moiety has a structure 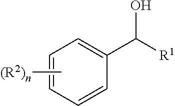
wherein
R¹ is H, aliphatic, aryl or heteroaryl;
each R₂ independently is aliphatic, aryl, heteroaryl, halogen, OH, NO₂, CO₂H, or ester; and
n is 0, 1, 2, 3, 4, or 5.

18. The method of claim 15, wherein the catalyst is present in the mixture in an amount of from 1 mol % to 10 mol %.

19. The method of claim 15, wherein the method further comprises separating the hydrogen peroxide from the oxidized product of the compound and any unreacted components from the mixture.

20. A method for producing hydrogen peroxide, the method comprising:
exposing a mixture comprising a compound comprising a benzylic alcohol moiety, 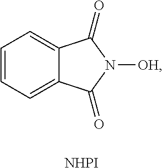 [SMILES: O=C1c2ccccc2C(=O)N1O ; "NHPI"]
and a photosensitizer to visible light at a temperature of 50° C. or less and in the presence of oxygen gas and in the absence of a metal, to form a reaction mixture comprising hydrogen peroxide; and
separating the hydrogen peroxide from the reaction mixture.
